(19) United States
(12) Patent Application Publication
(10) Pub. No.: US 2004/0049125 A1
(43) Pub. Date: Mar. 11, 2004

(54) Mobile terminal and mobile audiometer system

(76) Inventor: Norio Nakamura, Ibaraki (JP)

Correspondence Address: FISH & RICHARDSON P.C., 45 ROCKEFELLER PLAZA, SUITE 2800, NEW YORK, NY, 10111, US

(21) Appl. No.: 10/423,856

(22) Filed: Apr. 25, 2003

(30) Foreign Application Priority Data
Aug. 8, 2002 (JP) 2002-231149

(51) Int. Cl. A61B 5/00

(52) U.S. Cl. 600/559

(57) ABSTRACT

A mobile audiometer system is provided which enables a client to undergo an audiometric test anytime and anywhere using a mobile terminal such as a mobile phone without visiting a hospital and the like equipped with an audiometer. The mobile audiometer system downloads an audiometry program from an audiometry service providing apparatus to a mobile terminal such as a mobile phone or personal computer via a mobile network and the Internet, thereby enabling the client to have the audiometric test using the mobile terminal. The audiometry service providing apparatus can estimate gain correction values for a hearing compensation from the test results. The estimated values are downloaded to the mobile terminal for carrying out the fitting for the communication speech.

This application claims priority from Japanese Patent Application No. 2002-231149 filed Aug. 8, 2002, which is incorporated hereinto by reference.

### BACKGROUND OF THE INVENTION

1. Field of the Invention

The present invention relates to mobile information communication equipment such as mobile phones and personal computers (which are generically referred to as a “mobile terminal” in the present specification) and to an audiometric test system, and more particularly to a mobile terminal that operates as an audiometer, and to a mobile audiometer system configured by interconnecting a mobile terminal and an audiometry service providing apparatus via a network.

2. Description of the Related Art

FIG. 1 shows a conventional audiometry service providing apparatus, in which the wire of a headphone of a client is not physically connected to a diagnostic instrument (see, International Publication No. WO92/02172, for example). The audiometry service providing apparatus includes a control unit 20 and a remote unit 30. The control unit 20 includes a control console 21, an audiometer 22, a printer 23, a transmitter 24 and a receiver 25. On the other hand, the remote unit 30 includes a headphone 31, a transmitter 32, a receiver 33 and a hand pad 34.

To test a client, the transmitter 24 of the control unit 20 sends a signal to the headphone 31 of the remote unit 30. Listening to the signal, the client responds to it by pushing appropriate one of buttons 35 and 36 on the hand pad 34. Thus, the transmitter 32 of the remote unit 30 sends a response signal to the control unit 20. Receiving the signal from the remote unit 30, the receiver 25 delivers a demodulated response signal to the control console 21. In accordance with the incoming response signal, the control console 21 displays a test result on the audiometer 22.

Any person of an advanced age will face a problem of deteriorating the functions of body and sensory organs, and it is no exaggeration to say that in the super-aging society all the people are likely to have some kinds of handicaps. Generally, deterioration in hearing begins even from twenty years old, and clear hearing requires higher frequency components over 2 kHz. However, the deterioration with aging in the pure tone hearing ability becomes prominent above 2 kHz over fifty years old. Since the hearing deterioration progresses gradually, a lot of people are accustomed to a hard-of-hearing life without becoming aware of it, thereby delaying the detection of the hearing deterioration.

Although we should consider wearing hearing aids when we feel any impairment in hearing, the fact of matter is that wearing the hearing aids is usually delayed or unpopular. To start wearing hearing aids after checking the hearing deterioration, adaptation of listening is necessary because sound quality perceived with hearing aids at the current time differs from that of normal hearing. The adaptation is highly effective at an early stage of slight hearing loss, and therefore the detection of hearing problems at an earlier stage is important. To detect the hearing deterioration earlier, it is necessary to have a regular examination. The regular examination should be continued even after the detection, because the hearing will deteriorate with aging. Thus it is preferable that testers be spreading into individual homes to increase the chances of the tests. As for the conventional audiometer (corresponding to the audiometry service providing apparatus), it is necessary for a client of the audiometry to visit a hospital and the like equipped with an audiometer to undergo the test. In particular, the aged deteriorating in hearing must have the regular examination for suitably using hearing aids. However, since it is a demanding task for them to visit the hospital and the like, the hearing deterioration of the aged is ignored so that they are compelled to make an uncomfortable living.

In addition, as for the regular examination, it is inefficient to conduct precise audiometric tests for all the aged and future aged who are prolonging their life and whose number is increasing year after year. Not all the aged require a hearing aid. It is enough to screen only the persons who need the tests efficiently and economically. Then, only those who need more precise audiometric tests should undergo the tests. Thus, a tester is required for screening the hearing impaired more simply, conveniently and in a shorter time.

Such a screening test must satisfy absolute requirements such as it is consistent or repeatable and has universality. In other words, the tests must give the same result to each examiner regardless of time of the test. Besides, such an audiometric test preferably meets the following conditions: it requires no special skill, can be handled by an ordinary layman, is carried out simply in a short time, and can be screened by a user him or herself. In summary, it is ideal that any one can conduct the test conveniently as with thermometers spreading into almost all homes.

Furthermore, as typified by an e-government, the social life in the IT era is based on the premise that information equipment is utilized. Accordingly, the IT is likely to bring about digital divide between the information strong and information weak among the aged. The equipment expected to solve the problem is the ubiquitous computing. However, there are very few proposals and productions of barrier-free, easy-to-use ubiquitous equipment for the aged. For example, user friendly interfaces have been developed which utilize speech recognition and speech synthesis technology for the access to ubiquitous computers. However, since a greater number of the aged are not aware of their hearing deterioration and necessity of hearing aids, they do not wear the hearing aids, in which case there is a problem in that they cannot receive the advantages of the interfaces sufficiently.

Consequently, it is desired that flexible-personalized ubiquitous equipment suitable for the characteristics and contexts of users is implemented. To implement flexibility and personalization in the ubiquitous equipment, an audiometer capable of conducting audiometric tests conveniently anywhere and anytime is essential.

### SUMMARY OF THE INVENTION

The present invention is proposed to solve the foregoing problems of the conventional scheme. Therefore an object of the present invention is to provide a mobile terminal and mobile audiometer system enabling a client to undergo a hearing test readily anywhere and anytime by using a mobile phone or the like without going to a hospital equipped with an audiometer (corresponding to the audiometry service providing apparatus).

In addition, an object of the present invention is to facilitate automating regular periodic hearing tests, and storing and managing the test results.

According to a first aspect of the present invention, there is provided a mobile terminal including a computer program, the computer program causes the mobile terminal to carry out the steps of: giving instructions for an audiometric test to a user; and conducting the audiometric test of the user in accordance with responses of the user following the instructions.

Here, the mobile terminal may further comprise an earphone and a speaker, and the steps of giving instructions and conducting the audiometric test may be carried out using the earphone and speaker.

The computer program may cause the mobile terminal to carry out the further steps of: monitoring ambient noise around the mobile terminal; and making a decision as to whether surroundings of the mobile terminal are suitable for the audiometric test in accordance with the noise monitored.

The computer program may cause the mobile terminal to carry out the further steps of: storing results of the audiometric test; and compensating for speech output from the mobile terminal in accordance with the stored results of the audiometric test.

According to a second aspect of the present invention, there is provided a mobile audiometer system including an audiometry service providing apparatus having storing means that stores the computer program as defined in claim 1, and a mobile terminal connected to the audiometry service providing apparatus via a mobile network and the Internet, the mobile terminal comprising: download means for downloading the computer program from the audiometry service providing apparatus; and execution means for conducting an audiometric test of a user in accordance with instructions for an audiometric test given to the user and responses of the user following the instructions, by executing the computer program downloaded by the download means.

Here, the audiometry service providing apparatus may further comprise: acquisition means for acquiring results of the audiometric test from the mobile terminal; and electronic patient records for storing the results of the audiometric test acquired by the acquisition means.

The computer program may comprise: an instruction sub-program for causing the mobile terminal to carry out the instructions; and an audiometry sub-program for causing the mobile terminal to carry out the audiometric test.

The computer program may be created by the audiometry service providing apparatus.

The mobile terminal may further comprise an earphone and a speaker, and the instructions and the audiometric test may be carried out using the earphone and speaker.

The mobile terminal may further comprise: monitoring means for monitoring ambient noise around the mobile terminal; and decision means for making a decision as to whether surroundings of the mobile terminal are suitable for the audiometric test in accordance with the noise monitored by the monitoring means.

The audiometry service providing apparatus may further comprise means for regularly sending a request for an audiometric test to the mobile terminal.

The mobile audiometer system may further comprise a compensation unit for compensating for speech output from the mobile terminal in the audiometric test, and the compensation unit may comprise: download means for downloading the results of the audiometric test from the electronic patient records; and compensation means for compensating for the output speech in accordance with the results of the audiometric test downloaded by the download means.

The mobile terminal may further comprise: test result storing means for storing the results of the audiometric test; and compensation means for compensating for speech output from the mobile terminal in accordance with the results of the audiometric test stored in the result storing means.

More specifically, according to the present invention, the mobile terminal is utilized as the audiometer by downloading the audiometry program to the mobile terminal from the audiometry service providing apparatus on the Internet. Alternatively, the mobile terminal is utilized as the audiometer by installing the audiometry program into the mobile terminal. According to the present invention, the following advantages are offered:

(1) The mobile terminal does not require any special device or function for the audiometric test. This is because the test program is downloaded from the server to the mobile terminal, and the instruction sub-program with the instruction function and the audiometry sub-program with the audiometric test function are carried out on Java Virtual Machine or the like. In addition, when the test method is improved, the system can cope with it by only rewriting the program database on the server.

If the audiometer is implemented by utilizing the speech communication of the conventional fixed telephone or mobile phone to reproduce the test sounds, it will suffer from the noise of the communication network. However, utilizing the sound source or the like used for ringing tones, which is installed in the mobile phone, as in the present invention, makes it possible to reproduce low noise and stable test sounds. In addition, since the terminal is compact and portable, the test place can be changed easily. Any low noise environment found in a familiar place is applicable as a test room for a simple audiometric test.

(2) Since the audiometer in accordance with the present invention is not a specifically designed audiometer, it is usually carried about as a phone, and is used as a handy tester as needed. Accordingly, it is rare that the tester is not found when necessary.

(3) Configuring the audiometer at low cost utilizing the mobile phone will enable the individuals to readily possess the tester, which enables the testers to be spread into homes like thermometers. As a result, opportunities for the screening test increase, which will lead to early detection of the hearing deterioration and hearing impaired persons.

(4) Since the test results are stored in the server via the Internet, the system is appropriate for storing continuous test results, thereby facilitating construction of a database of the hearing characteristics of the aged. Thus, utilizing the previous audiometric test results stored can save the test time.

(5) The server computer automatically sends a request for regular periodic tests to the mobile terminal, and automatically stores the test results in the electronic patient records in the client database on the server computer. Therefore the client can undergo the regular periodic test without fail. In addition, the audiometry service providing apparatus offers an advantage of being able to make a hearing impairment assessment and fatigue test using the electronic patient records.

(6) The average hearing characteristics vary year after year in a super-aging society. In addition, the personal fitting to the hearing characteristics of a variety of individuals is an important problem. As for the design and development of products in the aging society, a database with a detailed and enormous amount of hearing characteristics is necessary. In this regard, the present invention is suitable for constructing the database of the hearing characteristics of the aged on the Internet, and for storing the test results continuously.

(7) An aged user can undergo the audiometric test anytime and anywhere as he or she likes, without going to a hospital or hearing aid store having an audiometry service providing apparatus.

(8) Monitoring the noise with a microphone in the mobile terminal such as a mobile phone or with an external microphone makes it possible to find an appropriate test place, without requiring any special test place as long as the place is a low noise environment.

The above and other objects, effects, features and advantages of the present invention will become more apparent from the following description of embodiments thereof taken in conjunction with the accompanying drawings.

### BRIEF DESCRIPTION OF THE DRAWINGS

FIG. 1 is a block diagram illustrating a configuration of a conventional audiometer;

FIG. 2 is a schematic diagram illustrating a configuration of a sound quality personal fitting system in a ubiquitous system;

FIG. 3 is a diagram illustrating a relationship between FIG. 3A and FIG. 3B;

FIG. 3A is a schematic diagram illustrating a configuration of a mobile audiometer system of a first embodiment in accordance with the present invention;

FIG. 3B is a schematic diagram illustrating the configuration of the mobile audiometer system of the first embodiment in accordance with the present invention;

FIG. 4 is a block diagram illustrating an exemplary operation of a mobile audio LSI executed by a program for an audiometric test written in Java language;

FIG. 5 is a diagram illustrating a relationship between FIG. 5A and FIG. 5B;

FIG. 5A is a schematic diagram illustrating a configuration of a mobile audiometer system of a second embodiment in accordance with the present invention;

FIG. 5B is a schematic diagram illustrating the configuration of the mobile audiometer system of the second embodiment in accordance with the present invention;

FIG. 6 is a schematic diagram illustrating a configuration of a mobile audiometer system of a third embodiment in accordance with the present invention; and

FIG. 7 is a schematic diagram illustrating a configuration of a mobile audiometer system of a fourth embodiment in accordance with the present invention.

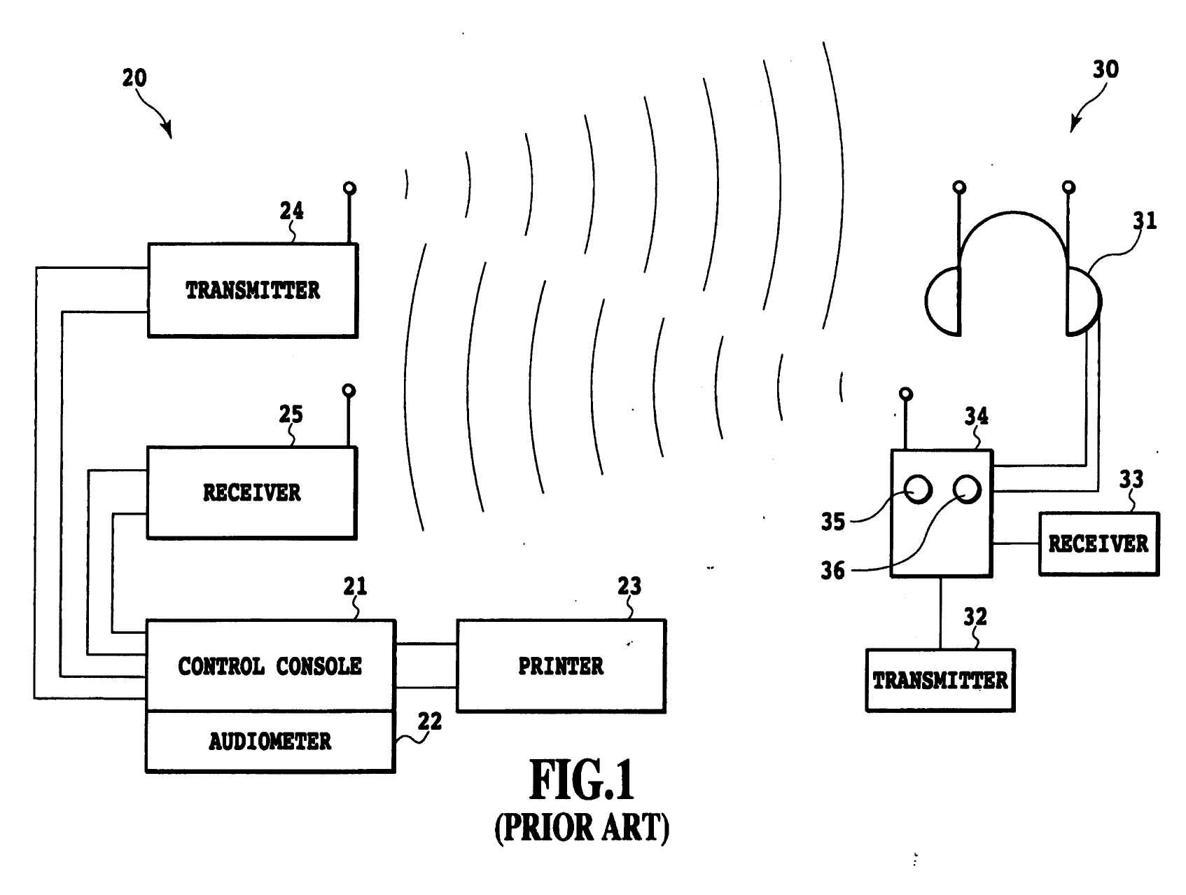

### DETAILED DESCRIPTION OF PREFERRED EMBODIMENTS

The embodiments in accordance with invention will now be described with reference to the accompanying drawings. Throughout the figures, the same reference numerals designate the same or like components.

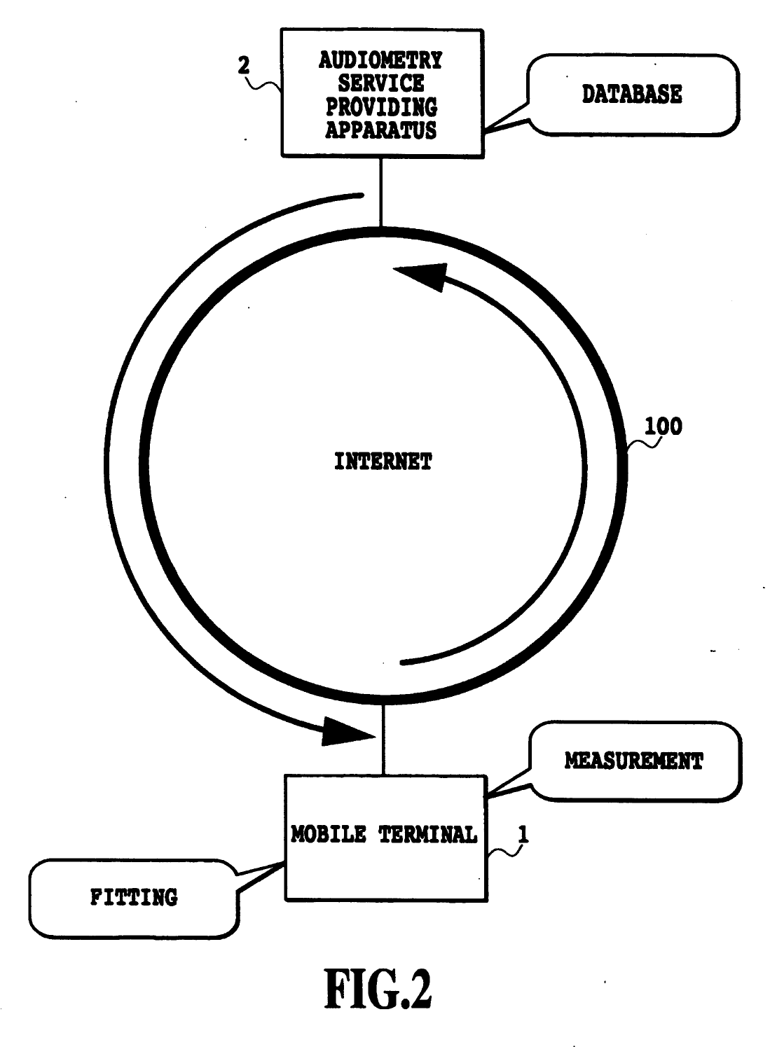

First, a system (called “sound quality personal fitting system” from now on) to which the present invention is applied will be described in its entirety with reference to FIG. 2. The sound quality personal fitting system in a ubiquitous system is composed of the following three phases: “measurement” for testing the hearing of a person; “database” for managing the test results; and “fitting” for carrying out personal fitting of the mobile terminal 1. In the “measurement”, an audiometric test is conducted using the mobile terminal 1 implemented in the form of a mobile phone, PDA, mobile personal computer, car navigation system and the like. In the “database”, the test results are stored as an electronic hearing chart in an audiometry service providing apparatus 2 such as a home server via the Internet 100. In the “fitting”, compensation values such as gain adjusted values are estimated from the test results for a compensation function. Then, the estimated values are downloaded into the mobile terminal 1, a fitting target, and whereby the sound quality fitting is carried out for the communication speech of the mobile terminal.

The following embodiments will be described by way of example of an audiometric test based on Japanese Industrial Standards (JIS). The ISO corresponding to JIS includes a standard for a pure tone audiometer, ISO 6189:1983 Acoustics—Pure tone air conduction threshold audiometry for hearing conservation purposes; a standard for pure tone audiometric test method, ISO 8253-1:1989 Acoustics—Audiometric test methods—Part 1: Basic pure tone air and bone conduction threshold audiometry; and a standard for a speech audiometric test method, ISO 8253-3:1996 Acoustics—Audiometric test methods—Part 3: Speech audiometry. In addition, as an audiometer for speech is known IEC 60645-2, Audiometers—Part 2: Equipment for speech audiometry. As for audiometric tests based on the other test standards such as ISO or IEC, it will become obvious that the present invention is applicable to these audiometric tests in accordance with the following embodiments.

### FIRST EMBODIMENT

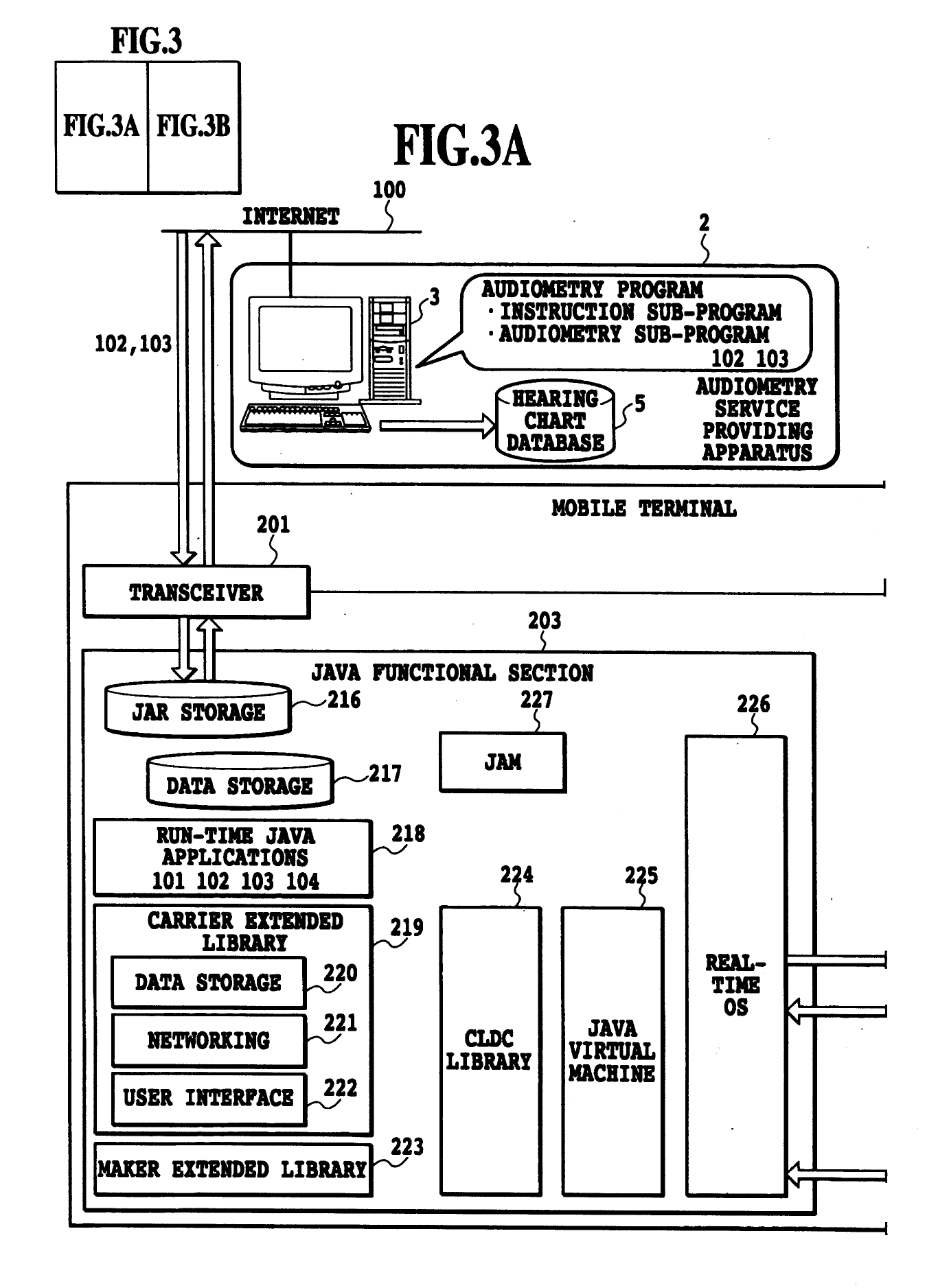

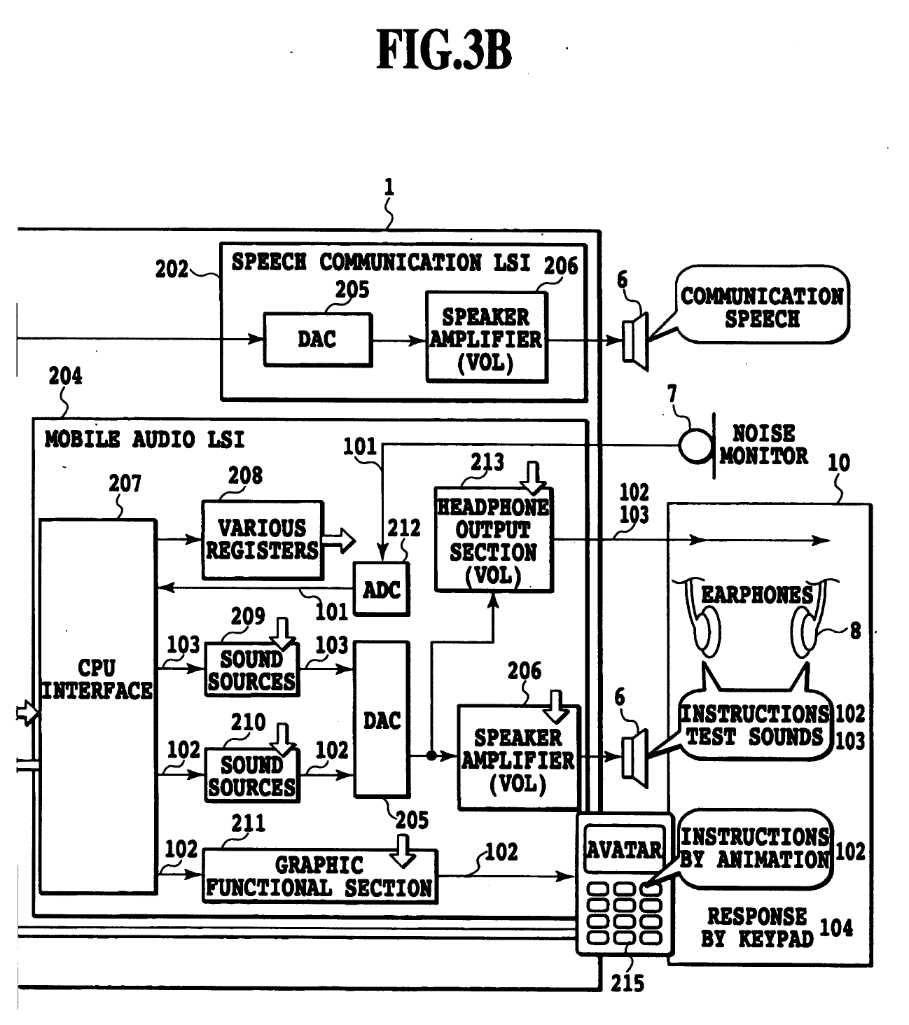

FIGS. 3A and 3B show a first embodiment of a mobile audiometer system. A mobile terminal 1 includes a speaker 6, a microphone 7, earphones 8 and a keypad 215. An audiometry service providing apparatus 2 includes a server computer 3 and electronic patient records 5 connected to the server computer 3, and is connected to the Internet 100.

Next, the configuration of the mobile terminal 1 will be described. The mobile terminal 1 includes a transceiver 201, a speech communication LSI 202, a Java functional section 203, and a mobile audio LSI 204.

The transceiver 201, being connected with the audiometry service providing apparatus 2 via a mobile network and the Internet 100, carries out the processings associated with the transmission and reception of data, with the modulation and demodulation of the data, and with the radio access scheme.

The speech communication LSI 202 includes a DAC 205 for converting the digital data on speech transmitted from the transceiver 201 to analog data, and a speaker amplifier 206 for supplying the speaker 6 with a speech signal in response to the analog data output from the DAC 205.

The Java functional section 203 includes a JAR storage 216 for storing JAR files which will be described later, a data storage 217 for storing data, run-time Java applications 218 consisting of execute-form files, a real-time OS 226, a JAM 227 for carrying out memory management and the like of a scratchpad, a Java virtual machine (Java VM) 225 for executing the Java applications and a variety of libraries. The libraries can include a CLDC library 224, a carrier extended library 219 and a maker extended library 223 as shown in FIG. 3A, though not limited to these libraries.

The scratchpad is utilized on the mobile terminal 1 as a storage area for storing data used by the applications.

The mobile audio LSI 204, which is provided for producing a ringing tone, includes a CPU interface 207, various registers 208, sound sources 209 and 210, a graphic functional section 211, an ADC (Analog/Digital Converter) 212, a DAC (Digital/Analog Converter) 205, a headphone output section 213, and a speaker amplifier 206. The CPU interface 207 is used by a CPU (not shown) which implements a function of the Java functional section 203 to exchange information with the real-time OS 226. The various registers 208 are used for the processing of the CPU. The ADC 212 converts ambient noise, which is received from the microphone 7 functioning as a noise monitor, to digital data. The sound source 209 is FM sound sources, and the sound source 210 is ADPCMs (PCMs) sound sources, both of which generate digital data on the ringer tones in accordance with the instructions from the CPU. The DAC 205 converts the digital data fed from the sound sources 209 and 210 to analog data. Receiving the analog data from the DAC 205, the headphone output section 213 outputs speech via the earphones 8, and the speaker amplifier 206 outputs the speech from the speaker 6. The graphic functional section 211 displays an image including an avatar on the display of the mobile terminal 1 in response following the instruction from the CPU. Here, the avatar refers to the other self of a tester, who operates in a virtual space of the computer, and can include text and speech besides the image.

In the present embodiment, the user who wishes to undergo the audiometric test, namely, the subject 10, takes an audiometric test by connecting the mobile terminal 1 to the Internet 100 via the mobile phone network, one of the mobile networks, and by downloading into the mobile terminal an audiometry sub-program from the audiometry service providing apparatus 2 connected to the Internet 100.

In FIGS. 3A and 3B, the reference numeral 101 designates a flow of noise measurement; the reference numeral 102 designates a flow of instructions by the avatar by using animation, text guidance and speech; the reference numeral 103 designates a flow of the audiometric test such as providing test sounds; and the reference numeral 104 designates a flow of responses of the subject.

Incidentally, the sound source for instructions whose flow is indicated by the reference numeral 102 can be used for reproducing masking sound at the test.

The audiometry program generated by the audiometry service providing apparatus 2 is stored in the storage in the server computer 3. As the storage, a nonvolatile memory such as a hard disk, DVD and CD-R can be used.

The audiometry program is downloaded from the audiometry service providing apparatus 2 into the mobile terminal 1 via the mobile phone network and the Internet 100. The computer program is downloaded by the communication based on the protocol such as ftp. The audiometry program is composed of an instruction sub-program for instructing a test method with the avatar using the animation, speech and text guidance, and the audiometry sub-program for providing the test sounds and for measuring the responses. The audiometry program gives the instructions of the test method, and carries out the audiometric test on the mobile terminal 1.

On the other hand, the responses of the subject 10 following the instructions and audiometric test, which are indicated by the reference numeral 104, are input from an operation key input section (keypad 215) of the mobile terminal 1.

The conventional audiometer is composed of a sine-wave generator, external signal source/masking noise generator, attenuator, response display, response unit, automatic recording/computer control audiometer, and equipment relating to the bone conduction. If comparisons are made between the main configuration of the conventional audiometer and that of the mobile audio LSI 204 used by the mobile terminal 1, there are following correspondences: the sine-wave generator corresponds to FM sound sources (sound source 209); the external signal source/masking noise generator corresponds to the ADPCM sound source (sound source 210); the attenuator corresponds to digital volumes (in headphone output section 213 and speaker amplifier 206); the response display corresponds to the display (graphic functional section 211); the response unit corresponds to the keypad 215; and the automatic recording/computer control audiometer corresponds to the Java application program 218. In other words, the present mobile terminal 1 and mobile audio LSI 204 used for the terminal are provided with the functions needed for the audiometer except for the performance associated with the bone conduction of the audiometer.

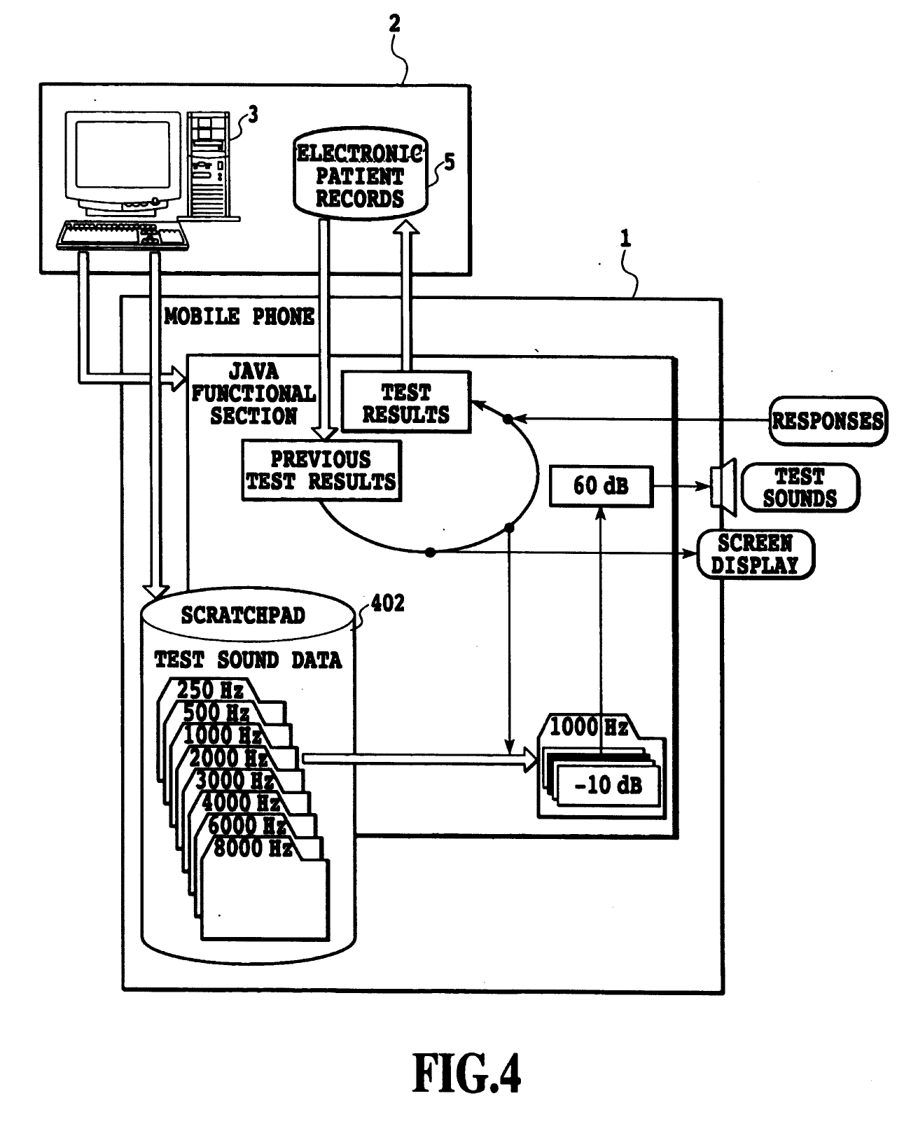

FIG. 4 illustrates the exemplary operation of the Java functional section. The JAR file consists of compressed class files and data files, and is stored in the scratchpad 402. In the present embodiment, the JAR file includes test sound data produced by using ringing tone data and arrivals at voice data which are usually utilized as ringing tones in the mobile terminal 1. The test sounds are produced from the maximum presentable sound pressure level to the minimum sound pressure level at every 5 dB step, and is compiled into folders for respective octave frequencies from 125 Hz to 8000 Hz. In the test, the folder corresponding to each test sound frequency is selected, and the file for the sound pressure level to be presented is selected in accordance with the test program.

As a presentation switch of the test sounds and a subject response system for the automatic test, the keypad 215 of the mobile terminal 1 is usable. In a manual test, the subject 10 selects the test sound with the keypad 215. In an automatic test, the test sound level is selected by the audiometric test function in accordance with the operation of the keypad 215 by the subject 10. The data on the test results is temporarily stored in the scratchpad memory, and the test results are displayed on the screen as the audiogram or the like after the test. The test results are uploaded into the server computer 3 on the network to be stored.

The audiometric test is conducted as follows. Although the following description is made by way of example of an audiometric test using the earphones, a test using the speaker or headphones can be carried out in the same manner.

(1) The client who wishes to undergo the audiometric test, namely, the subject 10, makes an access to the audiometry service providing apparatus 2 from his or her mobile terminal 1 to download the audiometry program for the audiometric test.

(2) When the mobile terminal 1 executes the audiometry program, the instructions on the test method are given by the avatar using the animation, speech and text guidance. For example, instructions such as “Are your ears cleaned? Don't you have otitis externa or eczema?”, “Haven't you heard excessively loud noise from 15 minutes ago?” and “Do you remove your hearing aid?” are given via the speaker 6. Listening and watching these instructions, the subject 10 makes a confirmation response by depressing a key [0].

(3) Subsequently, the audiometry program monitors the noise level for preparation of the test equipment and test environment. First, it issues the instructions with the avatar “The environment noise will be measured. Wait quietly for 10 seconds until the next instruction.” At the same time, the environment noise measured by the microphone 7 of the mobile terminal 1 is monitored by the noise measurement function, the flow of which is indicated by the reference numeral 101. If the requirement for the test environment is not satisfied, it is necessary for the client to move to a quieter place, or to undergo the test in nighttime hours. Nonetheless, if the environment is unsuitable for the audiometric test, a response is given that the test is impossible via the avatar. If the environment is suitable for the audiometric test, the instruction is issued such as “Sit down comfortably and take relaxed attitude.” to confirm the posture of the subject 10 through the avatar. The subject 10 depresses the key [0] as the confirmation.

(4) Next, the procedure of the test is instructed as follows with the avatar. For example, “Press the key [0] at once when you hear even the faintest test sound. Continue to press the key as long as you hear the test sound.” “Even if the sound is quiet, please respond without fail while the sound is audible.” “Release the key [0] at once when you no longer hear the test sound.” “Test your right ear, first. After the announcement of the end of the right ear test, test your left ear. The test sound increases its frequency such as 1000 Hz, 2000 Hz, 3000 Hz, 4000 Hz, 6000 Hz and 8000 Hz, and subsequently decreases its frequency such as 1000 Hz, 500 Hz, 250 Hz and 125 Hz.” “Press the key [1] to interrupt the test because of some circumstances. To resume the test after the interruption, press the key [3].” The subject 10 presses the key [0] as confirmation.

(5) Subsequently, instructions on wearing the earphones are given by the avatar. For example, “Wear the earphones on both your ears.” “Don't touch the earphones during the test.” After wearing the earphones 8 of the mobile terminal 1 on both ears, the subject 10 presses the key [0] as confirmation.

(6) Subsequently, instructions on preliminary test are given as follows with the avatar. “Then, let's start a preliminary test. Continue to press the key [0] as long as you hear the test sound. The test begins. If you are ready, press the key [0]. To interrupt the test, press the key [1]. To stop the test, press the key [7].” Then, the subject 10 presses the key [0] as confirmation.

After that, using the sound source 209, the audiometry sub-program reproduces the test sound of 1000 Hz and 40 dB via the earphones 8. If necessary, the masking noise is presented to the opposite ear via the earphones 8 by using the sound source 210. When a response is received from the subject 10, the test sound is reduced at 10 to 20 dB steps until no response is made. When no response is received, the test sound is increased at 10 to 20 dB steps until a response is made. Subsequently, the test sound is reduced until no response is made. Then, the level of the test sound is increased at 5 dB step to obtain the level at which a response is received at first. At the level at which the response of hearing is received at first, or at the level higher than that by 5 dB, a presentation and a halt of the test sound are repeated once or twice to check whether the presentation pattern of the test sound agrees with the response pattern. Then the end of the preliminary test is informed as follows with the avatar. “The preliminary test is completed successfully.” In contrast with this, instructions are given as follows, if necessary. “As a result of the preliminary test, we are afraid you don't understand the test method correctly. Listen to the explanation once again.”

The object of the preliminary test is to check roughly hearing levels of the subject 10 and to accustom the subject to the test method for the final test.

(7) Subsequently, the final test is begun. In the present embodiment, the final test is carried out according to the audiometric test method of JIS: To obtain the threshold, the pure tone audiometric test is conducted, and to test the actual hearing in everyday life, the above-threshold audiometric test is conducted. The above-threshold audiometric test is necessary to measure a phenomenon (recruitment phenomenon), in which a subjective sound-level abnormally changes as compared with normal hearing.

The final test is carried out by the audiometry sub-program. The final test will be described by way of example of the pure tone audiometric test.

First, the frequency of the test sound is set at 1000 Hz. Then its level is increased at every 5 dB step from the level 20 dB lower than the response level of the preliminary test. If necessary, the masking noise is presented to the opposite ear. The subject 10 presses the key [0] as a response when he or she can hear the test sound. The test sound is increased to 5-10 dB above the response level to make the subject confirm the test sound. Then the level of the test sound is reduced by 10-20 dB to check whether the same result can be obtained by the foregoing method. The subject 10 presses the key [0] as a response when he or she hears the test sound. If the response to the same level is received twice out of three time trials, the value is decided as the hearing level at 1000 Hz. If measurement values different by an amount equal to or greater than 15 dB are obtained in the three time trials, the procedure of the final test is iterated after repeating the explanation of the test. In this case, instructions are given as follows with the avatar. “As a result of the test, we are afraid you don't understand the test method correctly. Listen to the explanation once more.” The procedure of the final test is repeated after changing the frequency. When the test of the first ear has been completed, the test of the second ear is carried out in the same procedure. In this case, instructions are given as follows. “The test of your right ear has been completed. The test of your left ear is carried out next. If you are ready, press the key [0]”.

The measured hearing levels at respective frequencies are stored in the memory every time they are measured. When the entire test has been completed, the audiogram formed in a specified method is displayed on the screen of the mobile terminal 1. At the same time, information is given as follows with the test results on the screen. “The test has been completed. We report the test results.”

(8) The above-threshold audiometric test is conducted in the same manner.

(9) The test results are automatically written into the electronic patient records 5 in the audiometry service providing apparatus 2. Then, the audiogram and loudness curve which exhibit the hearing of the subject 10 are created, and the level and type of the hearing are decided. These items of information are stored as the test results.

(10) The audiometry service providing apparatus 2 is configured such that it transmits a request of the regular periodic test to the terminal via the mobile phone network and the Internet 100.

The speech audiometry consists of the speech reception test and the word discrimination test.

The speech reception test, which measures the threshold of the speech, is defined as a test of the faintest intensity (dB) at which “50% of correct answers” are obtained using easy-to-hear speech. The measurement results of the speech reception threshold are approximately equal to the average pure tone hearing level.

The word discrimination test examines the degree of distinguishing the speech correctly when the speech is presented at a sufficiently intelligible level above the threshold. The results of the word discrimination test are used to estimate the degree of impairment in the social life, and the estimation is used as an index of social adaptation or compensation. In addition, the test results are used to decide the possibility of wearing the hearing aids, to decide the better ear for wearing the hearing aids, and to decide the aided effect in the hearing aid fitting test. Furthermore, the test results play an important role in the evaluation of the effect after wearing an artificial ear. Thus, it is important to evaluate the hearing of the sensorineural deafness in particular.

In the word discrimination test, the sound source reproduces the test speech to obtain the response of the subject 10. If necessary, the masking noise is reproduced for the opposite ear. Before starting the test, appropriate instructions are given using the avatar. The instructions are given to the following items: Which ear is to be tested first; the type of the test speech; the method of responding (using the keys of the key pad); to respond even if the test speech is faintest, regardless of which ear hears the sound; to make a response immediately every time the subject hears the test speech; to respond just as it is heard even if it is uncertain; to ask a question without hesitation; and halting the test is allowed whenever sickened.

The speech reception test method will be described below.

As a speech table, JIS 57-S or 67-S is used. For example, the number list of the 67 speech table consists of seven rows, each of which includes six words.

The first row is used for the preliminary test. The instruction is give as “Continue to press the key [0] as long as you hear the sound”. Subsequently, the first speech sound in the first row is presented at a level sufficiently above the threshold, for example, at a level 40 dB (or 20 dB) above the average pure tone hearing level of the subject 10. As for the subsequent speech sounds, their intensity is reduced by 10 dB (or 5 dB) per sound by a descending method. Thus, the speech sound becomes inaudible to the subject in the course of the test. When the subject 10 becomes unable to hear, he or she releases the key [0] to halt the response. The preliminary test using the first row obtains the boundary between the audible level and inaudible level.

The final test is begun from the second row. The test of the second row is carried out after automatically adjusting the level of the first speech sound of the second row by the program such that the level of the boundary currently obtained is placed at the third or fourth speech sound. As with the six rows from the second to the seventh row, the test is carried out in the same manner so that the same columns of the respective rows each have the same sound intensity.

The total of 36 responses to the six rows by six columns beginning from the second row are automatically marked in terms of the percentage of correct answers for respective columns by the program, and are displayed on the screen as a speech audiogram.

Next, the word discrimination test method will be described.

As a speech table, the 57-S or 67-S is used.

The measurement is started from a level at which the speech is sufficiently audible. With changing a list and hearing level at 10-20 dB step, the client is requested to input hearing responses using the key. The program marks speech discrimination scores (intelligibility in terms of percent) automatically for each level, and the speech intelligibility curve is displayed on the speech audiogram.

The uncomfortable level measurement is conducted in the same manner as an air conduction audiometry by using an audiometer. An instruction is given by the avatar such as “Respond when you feel uncomfortable at the loudness”. The test sound (pure tone) is given for about three seconds at 5 dB steps, so that the subject responds when the test sound becomes uncomfortable. As soon as the client makes a response, the test sound is quieted. The same operation is repeated twice, and the latter measurement values are adopted as the results, which are written on the audiogram.

The test frequency is changed to 125 Hz, 250 Hz, 500 Hz, 1000 Hz, 2000 Hz, 3000 Hz, 4000 Hz, 6000 Hz and 8000 Hz. When the previous test results are stored in the electronic patient records 5, the test sound can be confirmed at a sufficiently audible level with reference to the previous test results. For example, the mobile terminal 1 can download the previous test results from the audiometry service providing apparatus 2 via the mobile network and the Internet 100, and stores them in the scratchpad 402. Subsequently, according to the stored test results, the test sound is reduced to a sound pressure level at which the test sound becomes inaudible. Subsequently, the sound intensity is increased by 5 dB at every one second interval, and the intensity level, at which the test sound becomes audible for the first time, is decided as the hearing level. The processing can reduce the time for the test.

The pure tone test sound can be replaced by narrow band noise.

The measurement of the comfortable level is carried out in the same manner as the air conduction audiometry for the measurement of the uncomfortable level using the audiometer. An instruction is given such as “Respond when the loudness is comfortable to hear”. The test sound (such as 1000 Hz pure tone) is increased from the threshold of the client at 5 dB steps at every five seconds interval, so that the client can respond when it is comfortable. An instruction is given such as “Release the response when you feel the sound slightly too loud”, so that the client stops the response when he or she feels the sound slightly too loud when the test sound is intensified at every 5 dB step at five second intervals. After the response, an instruction is given such as “Respond when the sound is just comfortable to hear”, followed by increasing the loudness of the test sound by 5 dB. Subsequently, the loudness of the test sound is reduced at 5 dB steps at five second intervals so that the client responds when the sound is just comfortable to hear (measurement value a). Then an instruction is given such as “Please stop the response when you feel the sound slightly too quiet”. The test sound is made quieter so that the client halts the response when he or she feels the sound slightly too quiet. After the response, an instruction is given such as “Respond when the sound is comfortable to hear”. Then, the sound intensity is further reduced by 5 dB, followed by increasing it again at 5 dB steps at five second intervals so that the client responds when the sound becomes just comfortable to hear (measurement value b). The average value of the measurement values a and b is decided as the comfortable level. When the first test is unreliable, the same procedure is repeated and the second result is adopted. If necessary, the tests at the frequencies 125 Hz, 250 Hz, 500 Hz, 1000 Hz, 2000 Hz, 3000 Hz, 4000 Hz, 6000 Hz and 8000 Hz are added.

### SECOND EMBODIMENT

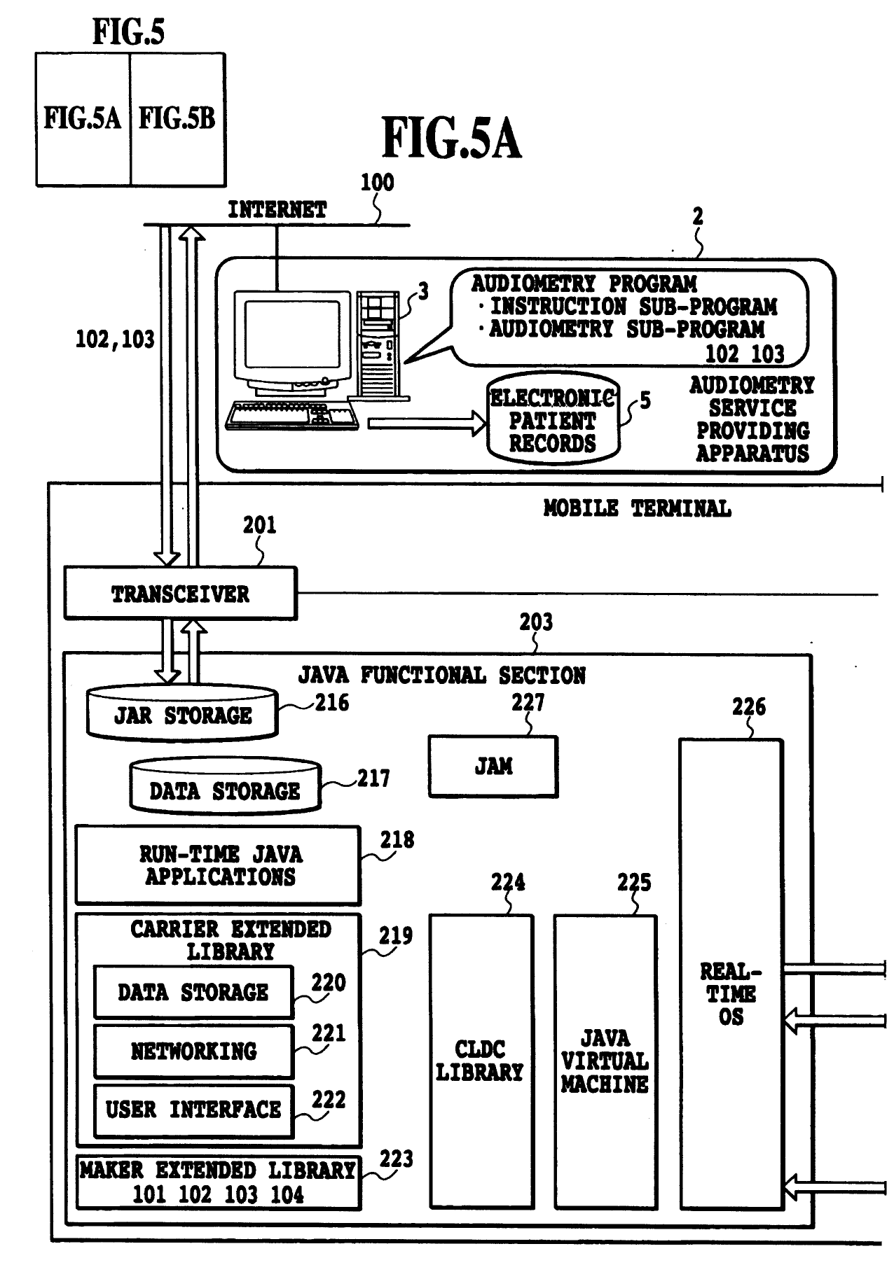

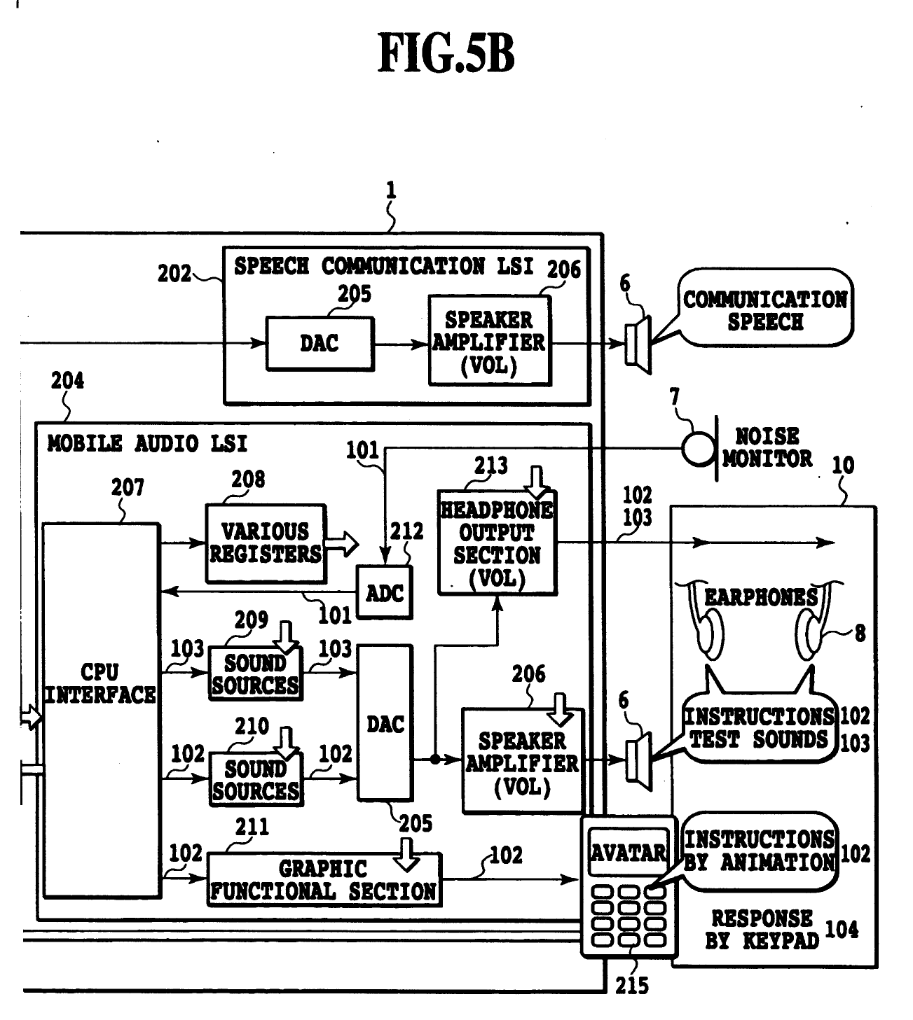

FIGS. 5A and 5B show a second embodiment of the mobile audiometer system. It differs from the first embodiment in that the audiometry program has already been installed in the mobile terminal with the remaining configuration being the same. In other words, the audiometry program has been installed into the mobile terminal 1 as a maker extended library in FIG. 5A or a circuit in the mobile audio LSI in FIG. 5B.

The audiometric test is carried out in the same manner as in the first embodiment.

### THIRD EMBODIMENT

In the following embodiments, the fitting of a mobile terminal using the results of the audiometric test will be described.

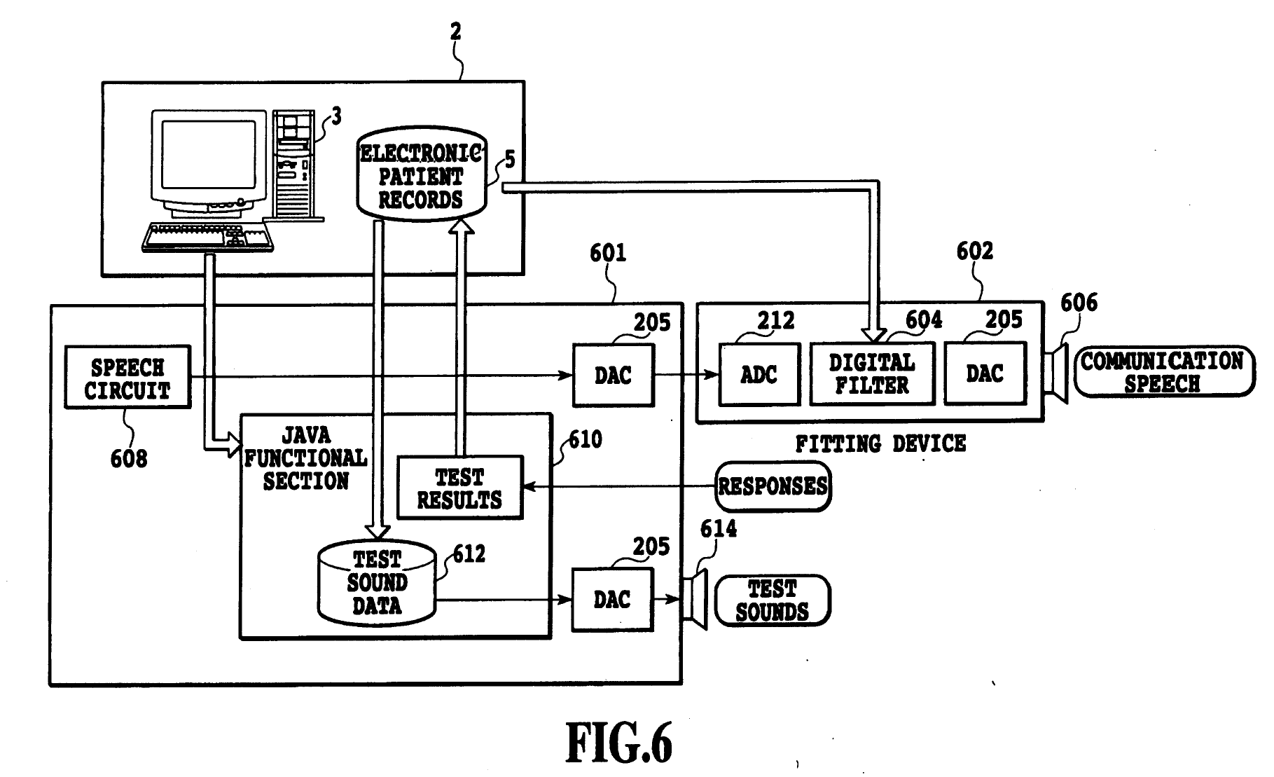

To design and develop a new mobile terminal for a hearing compensation will be difficult because of marketability and cost efficiency. Thus, as a practical solution, a method is adaptable which externally attaches a device with a hearing compensation (called “fitting device” from now on) to the mobile terminal as shown in FIG. 6.

As shown in FIG. 6, a mobile terminal 601 in accordance with the present embodiment includes a speech circuit 608, a Java functional section 610, an earphone 614 and DACs 205. The speech circuit 608 exchanges data with the audiometry service providing apparatus 2 via the mobile network and the Internet, and carries out modulation and demodulation of the data, and processing associated with a radio access scheme. The Java functional section 610 stores the test sound data 612. The DACs 205 convert the digital data of the ringer tone fed from the speech circuit 608 and the test sound data 612 into analog data, and output speech via the speaker and earphone.

The fitting device 602 includes an ADC 212, a digital filter 604, a DAC 205 and a speaker 606. These functional blocks are controlled by a controller such as a CPU (not shown) installed in the fitting device 602.

In an audiometric test phase, the Java functional section 610 uses the test sound data 612 to conduct the audiometric test by outputting the test sounds from the earphone 614 via the DAC 205. The test results are uploaded to the server computer 3.

In a fitting phase, the ADC 212 in the fitting device 602 converts the analog data of the communication speech, which is received from the mobile terminal 601, into the digital data. Then, the digital filter 604 compensates for the digital data, and the DAC 205 converts the compensated speech data into the analog data, and outputs the speech from the speech output section 606.

In the present embodiment, the server computer 3 designs the filter coefficients for compensating the communication speech in accordance with the hearing characteristics. In this case, the designed filter coefficients are downloaded from the server computer 3 to the programmable fitting device 602 to be used by the digital filter 604.

Alternatively, the fitting device 602 can carry out the filter design. In this case, such a configuration is possible in which the fitting device 602 downloads the test results stored in the electronic patient records 5 from the server computer 3, and designs the filter coefficients with reference to the test results.

The filter coefficients can also be designed in accordance with the characteristics of hardware constituting the speech output section 606 (speaker, earphone or headphone).

Furthermore, although the present embodiment is described by way of example of the mobile terminal that supplies the fitting device with the analog signal, it is also possible to use a mobile terminal that carries out digital input or output. In this case, the fitting device compensates for the digital signal fed from the mobile terminal, and returns the digital signal after the compensation to the mobile terminal. It is enough for the mobile terminal to convert the input digital signal to the analog signal, and to output the speech from the speaker or earphone for reproducing the analog signal.

### FOURTH EMBODIMENT

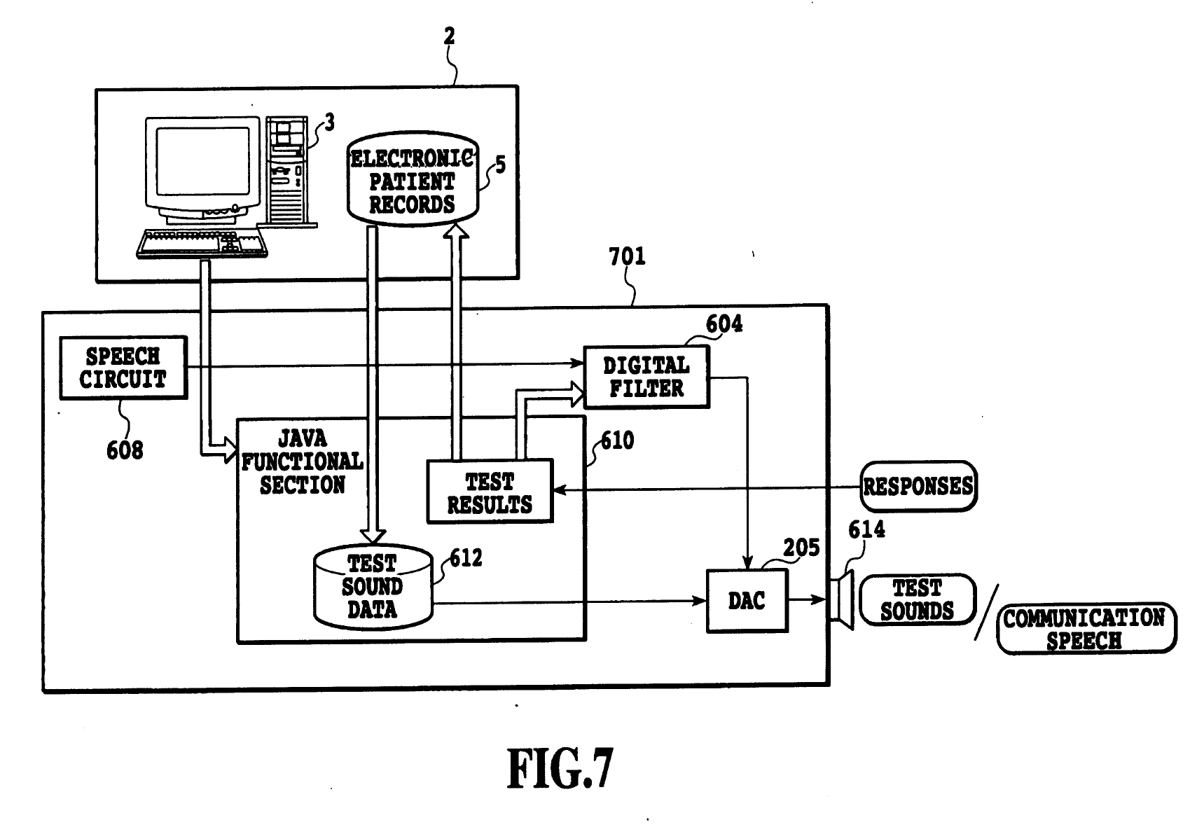

Besides the external-type fitting system, an integrated-type fitting system as shown in FIG. 7 is also possible. A mobile terminal 701 in accordance with the present embodiment includes a DAC 205, a speech circuit 608, a Java functional section 610, an earphone 614 and a digital filter 604.

In the audiometric test step, the Java functional section 610, using the test sound data 612, conducts the audiometric test by outputting the test sounds from the earphone 614 via the DAC 205. The Java functional section 610 stores the test results.

In the fitting step, the digital filter 604 compensates for the communication speech data using previous test results stored. Then, the DAC 205 converts the communication speech data passing through the compensation to analog data, and the earphone 614 outputs the speech.

Generally, as for the hearing aid fitting in accordance with the hearing test conducted by an audiometer, since a headphone specifically designed for the audiometer is used, the output of the hearing aids is different from that of the equipment to be fitted, thereby requiring conversion between them. In contrast with this, the hearing aid fitting using an SPL meter conducts an audiometric test using the same earphone as that of hearing aids. Thus, it is not necessary to obtain the compensating characteristics from the hearing characteristics and acoustic characteristics, making it possible to directly use the test results as the compensating characteristics without change.

The method of the present embodiment has the same advantage as the SPL meter in the fitting. More specifically, it uses the same sound source and earphone in both the aided condition and audiometric test situation for measuring the hearing with the mobile terminal for which the sound quality needs to be compensated. This will eliminate the need for the calibration for the fitting, thereby facilitating the fitting. To apply the measurement results of the hearing characteristics to the fitting of other equipment, however, the calibration is essential.

As the output means of the communication speech, other hardware such as a speaker or headphone can be used instead of the earphone. In this case, it is enough to design the filter coefficients in accordance with the hardware.

Although the size of the embedded hearing aid is a matter of concern for implementing the integrated-type personal fitting embedded in the mobile terminal as shown in FIG. 7, it is considered possible to utilize a small LSI chip used for a concha hearing aid.

Even if makers cannot cope with the integrated-type devices because of the marketability and cost efficiency, the attachable external-type devices described in the foregoing embodiment can be developed even by users themselves. If an open platform is provided which the users themselves can develop, the users themselves who know the need can improve the devices. In addition, exchanging information on the improvement and advantages will be effective.

The present invention has been described in detail with respect to preferred embodiments, and it will now be apparent from the foregoing to those skilled in the art that changes and modifications may be made without departing from the invention in its broader aspects, and it is the intention, therefore, in the appended claims to cover all such changes and modifications as fall within the true spirit of the invention.

What is claimed is:

1. A mobile terminal including a computer program, said computer program causes said mobile terminal to carry out the steps of:
giving instructions for an audiometric test to a user; and
conducting the audiometric test of the user in accordance with responses of the user following the instructions.

2. The mobile terminal as claimed in claim 1, further comprising an earphone and a speaker, wherein the steps of giving instructions and conducting the audiometric test are carried out using said earphone and speaker.

3. The mobile terminal as claimed in claim 1, wherein said computer program causes said mobile terminal to carry out the further steps of:
monitoring ambient noise around said mobile terminal; and
making a decision as to whether surroundings of said mobile terminal are suitable for the audiometric test in accordance with the noise monitored.

4. The mobile terminal as claimed in claim 1, wherein said computer program causes said mobile terminal to carry out the further steps of:
storing results of the audiometric test; and
compensating for speech output from said mobile terminal in accordance with the stored results of the audiometric test.

5. A mobile audiometer system including an audiometry service providing apparatus having storing means that stores the computer program as defined in claim 1, and a mobile terminal connected to said-audiometry service providing apparatus via a mobile network and the Internet, said mobile terminal comprising:
download means for downloading the computer program from said audiometry service providing apparatus; and
execution means for conducting an audiometric test of a user in accordance with instructions for an audiometric test given to the user and responses of the user following the instructions, by executing the computer program downloaded by said download means.

6. The mobile audiometer system as claimed in claim 5, wherein said audiometry service providing apparatus further comprising:
acquisition means for acquiring results of the audiometric test from said mobile terminal; and
electronic patient records for storing the results of the audiometric test acquired by said acquisition means.

7. The mobile audiometer system as claimed in claim 5, wherein said computer program comprises:
an instruction sub-program for causing said mobile terminal to carry out the instructions; and
an audiometry sub-program for causing said mobile terminal to carry out the audiometric test.

8. The mobile audiometer system as claimed in claim 7, wherein said computer program is created by said audiometry service providing apparatus.

9. The mobile audiometer system as claimed in claim 5, wherein said mobile terminal further comprises an earphone and a speaker, and wherein said instructions and said audiometric test are carried out using said earphone and speaker.

10. The mobile audiometer system as claimed in claim 5, wherein said mobile terminal further comprises:
monitoring means for monitoring ambient noise around said mobile terminal; and
decision means for making a decision as to whether surroundings of said mobile terminal are suitable for the audiometric test in accordance with the noise monitored by said monitoring means.

11. The mobile audiometer system as claimed in claim 5, wherein said audiometry service providing apparatus further comprises means for regularly sending a request for an audiometric test to said mobile terminal.

12. The mobile audiometer system as claimed in claim 5, further comprising a compensation unit for compensating for speech output from said mobile terminal in the audiometric test, said compensation unit comprising:
download means for downloading the results of the audiometric test from said electronic patient records; and
compensation means for compensating for the output speech in accordance with the results of the audiometric test downloaded by said download means.

13. The mobile audiometer system as claimed in claim 5, wherein said mobile terminal further comprises:
test result storing means for storing the results of the audiometric test; and
compensation means for compensating for speech output from said mobile terminal in accordance with the results of the audiometric test stored in said result storing means.
